(19) United States
(12) Patent Application Publication
(10) Pub. No.: US 2009/0098220 A1
(43) Pub. Date: Apr. 16, 2009

(54) LIQUID PHARMACEUTICAL PREPARATION COMPRISING FORSKOLIN FOR PROMOTING LEAN BODY MASS

(76) Inventor: Lance Ashworth, Ormond Beach, FL (US)

Correspondence Address: Jay M. Schloff;Intellipex PLLC, Suite 245, 30200 Telegraph Road, Bingham Farms, MI, 48025, US

(21) Appl. No.: 11/871,273

(22) Filed: Oct. 12, 2007

(51) Int. Cl. A61K 36/00; A61P 43/00

(52) U.S. Cl. 424/725

(57) ABSTRACT

A liquid pharmaceutical preparation comprising forskolin in an amount of about 0.025 mg/ml to about 1 mg/ml of the liquid pharmaceutical preparation for promoting lean body mass in a human subject. Further, an effective amount of the liquid pharmaceutical preparation comprising forskolin is administered subcutaneously in the human subject.

### FIELD OF THE INVENTION

The present invention relates generally to forskolin compositions, and, more particularly, to liquid pharmaceutical preparations comprising forskolin.

### BACKGROUND OF THE INVENTION

Forskolin is a natural compound obtained from Coleus forkohlii plant source. Forskolin has shown considerable therapeutic activity for promoting lean body mass and optimizing the body composition through cyclic AMP activation (promoting lipolysis) as well as increasing levels of hormone sensitive lipase.

Studies have been conducted in the past determining the lipolytic effect of forskolin. The study titled “body composition and hormonal adaptations associated with Forskolin consumption in overweight obese men”, Obesity Research 13:1335-1343 (2005), to Godard, M et al discusses the role of forskolin in the mobilization of the triglyceride stores that release fatty acids to be used for fuel within the body. Another study titled “Effects of Growth Hormone on the function of B-Adrenoceptor Subtypes in rat adipocytes”, Obesity Research 12:330-339 (2004), to Yang, S et al, discusses the determination of the lyoplytic activity of forskolin. Still, another study titled “Forskolin as an activator of cyclic AMP accumulation and lipolysis in rat adipocytes”, Molecular Pharmacology 22, 109-115 (1982), to Litoch, I et al, discusses the lipolytic effect of forskolin in vitro.

Forskolin compositions for promoting lean body mass are available in the art. Generally, such forskolin compositions are administered orally. The oral form is effective; however, unable to target specific deposits of fat in different parts of human body that would be addressed in an injectable form. Accordingly, what is needed is an injectable forskolin composition that may effectively target specific deposits of fat in different parts of the human body for promoting lean body mass.

### SUMMARY OF THE INVENTION

In view of the foregoing disadvantages inherent in the prior art, the general purpose of the present invention is to provide a liquid pharmaceutical preparation comprising forskolin for promoting lean body mass.

In a first aspect, the present invention provides a liquid pharmaceutical preparation comprising forskolin. The liquid pharmaceutical preparation is administered subcutaneously.

In another aspect, the present invention provides a method for promoting lean body mass in a human subject. The method comprises subcutaneously administering to the human subject an effective amount of a liquid pharmaceutical preparation comprising: forskolin; and at least one pharmaceutically acceptable carrier.

In another aspect, the present invention provides a method for preparing a liquid pharmaceutical preparation comprising forskolin. The method comprises: preparing a base vehicle; mixing forskolin powder with base vehicle; and adding sodium hydroxide solution for adjusting pH of the liquid pharmaceutical preparation to about 6.5.

These together with other aspects of the present invention, along with the various features of novelty that characterize the invention, are pointed out with particularity in the claims annexed hereto and form a part of this disclosure.

### DETAILED DESCRIPTION OF THE INVENTION

The exemplary embodiments described herein detail for a liquid pharmaceutical preparation. It is understood that various omissions and substitutions of equivalents are contemplated as circumstances may suggest or render expedient, but these are intended to cover the application or implementation without departing from the spirit or scope of the claims of the present invention. All ranges disclosed herein are inclusive and combinable.

The present invention provides a liquid pharmaceutical preparation comprising forskolin. The liquid pharmaceutical preparation of forskolin as mentioned herein includes an injectable preparation of forskolin to be used in dissolution of fatty tissue stores in specific areas of the body, including the subcutaneous fat on abdominals, thighs, hips, and other areas having fatty tissue stores.

Preferably, the liquid pharmaceutical preparation of the present invention is administered subcutaneously. Such an administration of the liquid pharmaceutical preparation increases the ratio of the lean body mass to the subcutaneous fat in the areas of administration.

Without being bound by any theory, the liquid pharmaceutical preparation of the present invention promotes an increase in levels of hormone sensitive lipase (HSL) and adenylate cyclase within adipocytes. HSL promotes conversion of triglycerides to fatty acids that are more readily available for energy production by surrounding tissues, once liberated from the adipocyte. The increase in levels of adenylate cyclase results in an increase in levels of cyclic AMP and promotes lipolysis. Such mechanism provides the necessary liberation of fat stores, causing fat cells to decrease in size, thereby promoting lean body mass.

The liquid pharmaceutical preparation may comprise forskolin in an amount of about 0.001 milligram/milliliter (mg/ml) to about 5 mg/ml of the liquid pharmaceutical preparation, or, more specifically, in an amount of about 0.01 mg/ml to about 4 mg/ml of the liquid pharmaceutical preparation, or, even more specifically, in an amount of about 0.025 mg/ml.

Drug dosage and frequency of administration depend on efficacy and tolerance. In accordance with the present invention, the dosage of the liquid pharmaceutical preparation that is administered to a subject is effective to achieve the desired result of promoting the lean body mass in the subject. Additionally, the dosage is determined by the specific area of the body of the subject in which the fat is targeted. Preferably, the daily dose is of about 20 ml to about 30 ml for targeting the fat in the abdominals of the subject, of about 30 ml to about 50 ml for targeting the fat in the lateral thighs, of about 5 ml to about 15 ml for targeting the fat in the inner thighs, and of about 15 ml to about 30 ml for targeting the fat on the back portion of the body. The dose may be administered once a day and repeated no more than once every three weeks.

The liquid pharmaceutical preparation may further comprise at least one pharmaceutically acceptable carrier or diluent, wherein the pharmaceutically acceptable carrier is selected from the group consisting of solvents, buffers, stabilizers, antioxidants, chelating agents, wetting agents, solubilizing agents, tonicity modifiers, preservatives, and combinations comprising at least one of the foregoing.

For the liquid pharmaceutical preparation, suitable solvents include, but are not limited to, water, polyethylene glycol, trimethylene glycol, 1, 2-propane diol, N, N-dimethyl acetalide, and ethanol. Preferably, sterile water may be used as a diluent for solubilization.

The liquid preparation may include one or more buffers. Suitable buffers include, but are not limited to, citrate buffer, phosphate buffer (for example, sodium phosphate monobasic), acetate buffer, carbonate buffer, and triethanolamine buffer.

The stabilizer for the liquid pharmaceutical preparation may be a metal salt additive. Suitable metal salt additives, such as, calcium carbonate, calcium hydroxide, magnesium carbonate, magnesium hydroxide, magnesium silicate, magnesium aluminate, and aluminum magnesium hydroxide, sodium carbonate and disodium hydrogen orthophosphate, and the like, may be used as stabilizer of forskolin.

The pharmaceutically acceptable antioxidants may be selected from amongst one or more of the suitable antioxidants known in the art. Examples of suitable pharmaceutically acceptable antioxidants include, but are not limited to, butylated hydroxyanisole (BHA), sodium ascorbate, butylated hydroxytoluene (BHT), sodium sulfite, citric acid, malic acid, and ascorbic acid.

Chelating agents may also be used, such as, for example, diamine tetraacetic acid (EDTA) or diethylenetriamine pentaacetic acid (DTPA), or a suitable salt thereof.

Suitable wetting agents that may be used include, but are not limited to, sodium lauryl sulfate, docusate sodium, ethoxylated castor oil, a polyglycolyzed glyceride, an acetylated monoglyceride, a sorbitan fatty acid ester, a poloxamer, a polyoxyethylene sorbitan fatty acid ester, a polyoxyethylene, a monoglyceride and ethoxylated derivatives thereof, a diglyceride and ethoxylated derivatives thereof, and mixtures thereof.

As used herein, a “solubilizing agent” refers to a substance that is able to enhance the solubility of the lyophilized cake on reconstitution with physiological solutions. Useful solubilizing agents include, but are not limited to, L-arginine, histidine, and lysine.

Tonicity modifiers may also be used for adjusting the isotonicity of the liquid pharmaceutical preparation. Useful tonicity modifiers include, but are not limited to, glycerin, sodium chloride, maltose, mannitol, dextrose and mixtures thereof.

Similarly, suitable preservatives for the liquid pharmaceutical preparation of the present invention may include, but are not limited to, methyl hydroxy benzoic acid, propyl hydroxy benzoic acid, phenol, benzyl alcohol, sodium benzoate, and edentate calcium disodium.

Further, the present invention provides a method for promoting lean body mass in a human subject. The method comprises subcutaneously administering to the human subject a therapeutically effective amount of a liquid pharmaceutical preparation comprising forskolin and at least one pharmaceutically acceptable carrier.

The method further comprises concurrent administration of at least one other mesotherapy agent. As used herein, the term ‘concurrent administration’ refers to the administration of a simple physical mixture (also referred to as ‘concurrent administration mixture’) prepared by combining the liquid pharmaceutical preparation and at least one other mesotherapy agent.

Suitable mesotherapy agents include, but are not limited to, phosphatidylcholine, aminophylline, isoproterenol, hyaluronidase, collagenase, and lidocaine.

In one embodiment, the method for promoting lean body mass in a human subject comprises concurrent administration of phosphatidylcholine, aminophylline, isoproterenol, and lidocaine, along with the liquid pharmaceutical preparation comprising forskolin and normal saline. This embodiment is particularly useful for fat dissolving or body sculpting. In another embodiment, the method for promoting lean body mass in a human subject comprises concurrent administration of aminophylline, hyaluronidase, and collagenase, along with the liquid pharmaceutical preparation comprising forskolin. This embodiment is particularly useful for cellulite applications used for the thighs and buttocks.

The present invention further provides a method for preparing a liquid pharmaceutical preparation comprising forskolin. The method comprises: preparing a base vehicle; mixing forskolin powder with the base vehicle; and adding sodium hydroxide solution for adjusting the pH of the liquid pharmaceutical preparation to about 6.5.

The present invention is further illustrated by the following non-limiting examples.

### EXAMPLE 1

This example describes the method for preparing a liquid pharmaceutical composition comprising forskolin. The example further describes the method of administration of the liquid pharmaceutical preparation.

The components in the base vehicle solution and respective quantity of each component are shown in Table 1.

TABLE 1

| Ingredients | Quantity | |
|---|---|---|
| Sodium chloride | 1.0625 | gm |
| Calcium chloride | 0.0664 | gm |
| Edentate calcium disodium | 0.1000 | gm |
| Sodium phosphate monobasic | 0.2112 | gm |
| Sterile water for injection | q.s. 100 | ml |

The base vehicle solution was prepared by dissolving all the powders of sodium chloride, calcium chloride, edentate calcium disodium, sodium phosphate monobasic in sterile water for injection. Next, 2.5 mg of high purity (99.78%) forskolin was mixed with the base vehicle solution. The preparation was left for 30 minutes. The pH of the preparation was adjusted to 6.5 by adding 10 percent sodium hydroxide solution. The preparation was allowed to spin for at least 6 hours in refrigerated conditions. The final pH was checked and adjusted, if necessary. Next, the preparation was filtered into sterile vials using 0.22 micrometer super filters, to produce 98% pure, standardized root extract. Forskolin was present in an amount of about 0.025 mg/ml of the liquid pharmaceutical preparation.

5 ml of the liquid pharmaceutical preparation (0.025 mg/ml forskolin) was added to 5 ml of normal saline in a 10 ml syringe. The 10 ml preparation was subcutaneously administered at a depth of 6 millimeter (mm) using a 6 mm, 30 gauge needle. The administration was spaced out in a point to point manner in 0.75 inch intervals along the area containing the subcutaneous fat to be dissolved. Such a method of administering the preparation resulted in an increase in the ratio of the lean body mass to the subcutaneous fat in the area in which the preparation was administered.

### EXAMPLE 2

This example describes the composition of a concurrent administration mixture for the concurrent administration of the liquid pharmaceutical preparation of Example 1 along with phosphatidylcholine, aminophylline, isoproterenol, and lidocaine. This example further describes the method of administration of the concurrent administration mixture.

1.5 ml of the liquid pharmaceutical preparation (0.025 mg/ml forskolin) was added to a 10 ml syringe. To this 10 ml syringe, the following were added: 2 ml of 100 mg/ml of phosphatidylcholine, 3 ml of 25 mg/ml of aminophylline, 0.5 ml of about 32 picomoles/ml isoproterenol, 2 ml of normal saline, and 1 ml of 2% lidocaine, to form a 10 ml preparation.

The 10 ml preparation was administered subcutaneously at a depth of 6 millimeter (mm) using a 6 mm, 30 gauge needle. The administration was spaced out in a point to point manner in 0.75 inch intervals along the area containing the subcutaneous fat to be dissolved. Such a method of administering the preparation resulted in an increase in the ratio of the lean body mass to the subcutaneous fat in the area in which the preparation was administered.

### EXAMPLE 3

This example describes the composition of a concurrent administration mixture for the concurrent administration of the liquid pharmaceutical preparation of Example 1 along with aminophylline, hyaluronidase, and collagenase. This example further describes the method of administration of the concurrent administration mixture.

2 ml of the liquid pharmaceutical preparation (0.025 mg/ml forskolin) was added to a 10 ml syringe. To this 10 ml syringe, the following were added: 3 ml of 25 mg/ml of aminophylline, 4 ml of 150 units/ml hyaluronidase, 1 ml of 1000 units/ml collagenase, to form a 10 ml preparation.

The 10 ml preparation was administered subcutaneously at a depth of 4 millimeters (mm) using a 4 mm, 30 gauge needle. The administration was spaced out in a point to point manner in 0.25 inch intervals along the area containing the subcutaneous fat to be dissolved. The amount injected at each point was about 0.2 ml. Such a method of administering the preparation (also referred to as cellulite preparation) resulted in an increase in the ratio of the lean body mass to the subcutaneous fat in the area (thighs and buttocks) in which the preparation was administered.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, and to thereby enable others skilled in the art to best utilize the invention and various embodiments with various modifications as are suited to the particular use contemplated. It is understood that various omissions and substitutions of equivalents are contemplated as circumstances may suggest or render expedient, but these are intended to cover the application or implementation without departing from the spirit or scope of the claims of the present invention.

What is claimed is:

1. A liquid pharmaceutical preparation for promoting lean body mass in a human subject, the liquid pharmaceutical preparation comprising forskolin in an amount of about 0.025 mg/ml to about 1 mg/ml of the liquid pharmaceutical preparation.

2. The liquid pharmaceutical preparation of claim 1, wherein the liquid pharmaceutical preparation is administered subcutaneously.

3. (canceled)

4. The liquid pharmaceutical preparation of claim 1, further comprising at least one of solvents, buffers, stabilizers, antioxidants, chelating agents, wetting agents, solubilizing agents, tonicity modifiers, preservatives, and combinations comprising at least one of the foregoing.

5. (canceled)

6. A method for promoting lean body mass in a human subject, the method comprising subcutaneously administering to the human subject a therapeutically effective amount of the liquid pharmaceutical preparation of claim 1.

7. (canceled)

8. The method of claim 6, further comprising concurrent administration of at least one other mesotherapy agent.

9. The method of claim 8, wherein the at least one other mesotherapy agent is selected from the group consisting of aminophylline, isoproterenol, hyaluronidase, collagenase, and lidocaine.

10. The method of claim 8, further comprising concurrent administration of aminophylline, isoproterenol, and lidocaine.

11. The method of claim 8, further comprising concurrent administration of aminophylline, hyaluronidase, and collagenase.

12. A method for preparing the liquid pharmaceutical preparation of claim 1, the method comprising:
preparing a base vehicle;
mixing forskolin powder with the base vehicle; and
adding sodium hydroxide solution for adjusting pH of the liquid pharmaceutical preparation to about 6.5.

13. The method of claim 12, wherein the base vehicle comprises sodium chloride, calcium chloride, edentate calcium disodium, sodium phosphate monobasic, and water.

14. (canceled)
